(19) United States
(12) Patent Application Publication
(10) Pub. No.: US 2024/0374886 A1
(43) Pub. Date: Nov. 14, 2024

(54) INTRODUCER SHEATH WITH DUAL ARM HUB HAVING DETACHABLE TIGHTENING PORT

(71) Applicants: Boston Scientific Medical Device Limited, Galway (IE); Boston Scientific Scimed, Inc., Maple Grove, MN (US)

(72) Inventors: Neeraj Kumar Sharma, Delhi (IN); Qian Liu, Plymouth, MN (US); Peeyush Tomar, Muzaffarnagar (IN)

(73) Assignees: Boston Scientific Medical Device Limited, Galway (IE); Boston Scientific Scimed, Inc., Maple Grove, MN (US)

(21) Appl. No.: 18/659,556

(22) Filed: May 9, 2024

Related U.S. Application Data
(60) Provisional application No. 63/465,984, filed on May 12, 2023.

(51) Int. Cl. A61M 39/06; A61M 25/00

(52) U.S. Cl. CPC A61M 39/06; A61M 25/0097; A61M 2039/0626; A61M 2039/0633

(57) ABSTRACT

An introducer sheath including a valve hub and an elongate shaft extending from the valve hub. The valve hub defines a main port and a side port. The main port includes a hub body, a seal disposed within the hub body, a main port lid attached to the hub body, a tightening port hub attached to the main port lid, a compressible seal disposed within a lumen of the tightening port hub, a pusher at least partially positioned within the lumen of the tightening port hub and slidably movable relative thereto, and a lock nut surrounding the pusher. The lock nut is threadably engaged with the tightening port hub such that rotation of the lock nut moves the pusher axially toward and/or away from the compressible seal.

### CROSS REFERENCE TO RELATED APPLICATIONS

This application claims the benefit of U.S. Provisional Patent Application Ser. No. 63/465,984, filed May 12, 2023, which is incorporated herein by reference.

### TECHNICAL FIELD

The present disclosure pertains to sheaths for delivering intravascular medical devices. More specifically, the present disclosure relates to a hub of a sheath with a tightening port for securing a medical device, such as a catheter or blood pump, within the hub and thus fix the position of the catheter with respect to the hub and sheath.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters or other devices, such as a blood pump. A hub may be incorporated at a proximal end of the sheath to provide access to a lumen of an elongate shaft of the sheath. The hub may include one or more seals, e.g., hemostasis seals, to reduce blood leakage as devices are being inserted, positioned, and removed. In various instances, there may be a desire to fix the position of the medical device within the sheath and vasculature. Additionally, there may be a desire to reposition the medical device within the vasculature. Thus, there is a need for improved hub configurations for a sheath including sealing mechanisms to reduce blood leakage past the medical device and/or tightening mechanisms for securing the medical device, such as a catheter or blood pump, within the sheath and vasculature that also allow repositioning of the medical device in the vasculature.

### BRIEF SUMMARY

This disclosure provides design, material, manufacturing method, and use alternatives for medical devices, including introducer and/or repositioning sheaths.

A first example is directed to an introducer sheath including a valve hub and an elongate shaft extending from the valve hub. The valve hub defines a main port and a side port. The main port includes a hub body, a seal disposed within the hub body, a main port lid attached to the hub body, a tightening port hub attachable to the main port lid, a compressible seal disposed within a lumen of the tightening port hub, a pusher at least partially positioned within the lumen of the tightening port hub and slidably movable relative thereto, and a lock nut surrounding the pusher. The lock nut is threadably engaged with the tightening port hub such that rotation of the lock nut moves the pusher axially toward and/or away from the compressible seal.

Alternatively or additionally to any of the examples above, in another example, the pusher includes a flange extending radially from a body of the pusher.

Alternatively or additionally to any of the examples above, in another example, an internal rim of the lock nut engages the flange.

Alternatively or additionally to any of the examples above, in another example, the main port lid is threadably engaged with the hub body of the main port.

Alternatively or additionally to any of the examples above, in another example, the main port lid includes first and second tabs extending radially therefrom.

Alternatively or additionally to any of the examples above, in another example, the tightening port hub includes first and second slots or notches for receiving the first and second tabs, respectively.

Alternatively or additionally to any of the examples above, in another example, the lock nut is threadably engaged with a threaded stem of the tightening port hub.

Alternatively or additionally to any of the examples above, in another example, the pusher includes an anti-rotation tab slidably disposed in a channel of the tightening port hub.

Alternatively or additionally to any of the examples above, in another example, a distal end region of the pusher that is positioned within the lumen of the tightening port hub includes an enlarged diameter rim, wherein the enlarged diameter rim has a larger diameter than a portion of the lumen located proximal thereof.

Alternatively or additionally to any of the examples above, in another example, the introducer sheath includes a sleeve gripper surrounding a proximal end region of the pusher.

Another example is a medical device assembly. The medical device assembly includes a medical device having an elongate shaft and a tightening port attachable to a hub of an introducer sheath. The tightening port includes a tightening port hub, a compressible seal disposed within a lumen of the tightening port hub, a pusher at least partially positioned within the lumen of the tightening port hub and slidably movable relative thereto, and a lock nut surrounding the pusher. The lock nut is threadably engaged with the tightening port hub such that rotation of the lock nut moves the pusher axially toward and/or away from the compressible seal. The medical device assembly also includes a sterile sleeve surrounding the elongate shaft such that the elongate shaft is moveable axially relative to the sterile sleeve through a lumen of the compressible seal. A distal end of the sterile sleeve is coupled to the tightening port.

Alternatively or additionally to any of the examples above, in another example, the distal end of the sterile sleeve is secured to the pusher.

Alternatively or additionally to any of the examples above, in another example, a spindle of the pusher is inserted into a lumen of the sterile sleeve and a collar surrounds the distal end of the sterile with the distal end of the sterile sleeve clamped between the collar and the spindle.

Alternatively or additionally to any of the examples above, in another example, a distal end region of the pusher that is positioned within the lumen of the tightening port hub includes an enlarged diameter rim, wherein the enlarged diameter rim has a larger diameter than a portion of the lumen located proximal thereof.

Alternatively or additionally to any of the examples above, in another example, the pusher includes a flange extending radially from a body of the pusher and an internal rim of the lock nut engages the flange.

Another example is a method of inserting a medical device into a body of a patient. The method includes intraoperatively inserting an elongate shaft of the medical device through an elastomeric seal in a hub of an introducer sheath and into a vasculature of the patient. Thereafter, the method includes coupling a tightening port to the hub of the introducer sheath after inserting the elongate shaft through the elastomeric seal.

Alternatively or additionally to any of the examples above, in another example, the elongate shaft extends through a compressible seal of the tightening port as the elongate shaft of the medical device is inserted through the elastomeric seal in the hub of the introducer sheath and into the vasculature of the patient.

Alternatively or additionally to any of the examples above, in another example, the tightening port includes a tightening port hub, a compressible seal disposed within a lumen of the tightening port hub, a pusher at least partially positioned within the lumen of the tightening port hub and slidably movable relative thereto, and a lock nut surrounding the pusher. The lock nut is threadably engaged with the tightening port hub such that rotation of the lock nut moves the pusher axially toward and/or away from the compressible seal.

Alternatively or additionally to any of the examples above, in another example, the method includes tightening the compressible seal around the elongate shaft of the medical device with the lock nut after coupling the tightening port to the hub of the introducer sheath.

Alternatively or additionally to any of the examples above, in another example, a sterile sleeve having a distal end coupled to the tightening port is provided. The sterile sleeve extends proximally from the tightening port and surrounds the elongate shaft such that the elongate shaft is moveable axially relative to the sterile sleeve.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a side view of an introducer sheath extending into a blood vessel;

FIG. 2 is a cross-sectional view of a portion of the introducer sheath of FIG. 1 inserted into a blood vessel, and a medical device inserted into the introducer sheath;

FIG. 3 is a perspective view of the introducer sheath of FIG. 1;

FIG. 4 is a side view of various components of the introducer sheath of FIG. 3;

FIG. 5 is an exploded side view of various components of the introducer sheath of FIG. 3;

FIG. 6 is an exploded perspective view of various components of the introducer sheath of FIG. 3;

FIG. 7 is a cross-sectional view of various components of the introducer sheath of FIG. 3;

FIG. 8 is a cross-sectional view of various components of the introducer sheath of FIG. 3;

FIG. 9 is a cross-sectional view of the introducer sheath of FIG. 3, with a tightening port provided with a medical device and detached from the hub body of the main port of the introducer sheath;

FIG. 10 is a cross-sectional view of the introducer sheath of FIG. 3 with the tightening port attached to the hub body of the main port of the introducer sheath and a medical device passing through the hemostasis valve in an open position; and

FIG. 11 is a cross-sectional view of the introducer sheath of FIG. 3 with the tightening port attached to the hub body of the main port of the introducer sheath and a medical device passing through the hemostasis valve in a closed position.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Hemostasis valve hub assemblies provided with an introducer sheath facilitate insertion and positioning of one or more medical devices (e.g., catheters, blood pumps, guidewires, etc.) through the introducer sheath while also helping to prevent blood from leaking during a medical procedure. Some embodiments of the present disclosure feature assemblies with components that assist with fixing the position of one or more medical devices while maintaining a seal around the elongate shaft of the medical device to reduce blood loss through the hub assembly. Further, some embodiments may also provide components that facilitate convenient repositioning of the one or more medical devices relative to the introducer sheath.

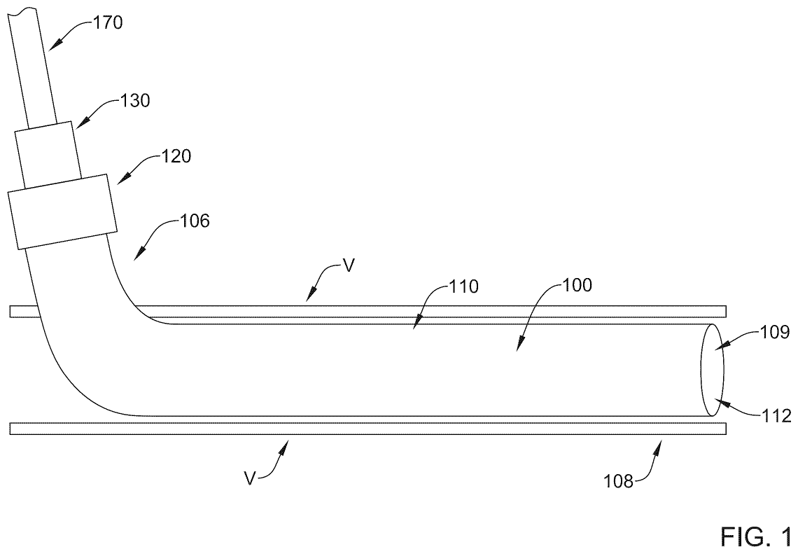

FIG. 1 illustrates a side view of an introducer sheath 100 inserted at least partially into a blood vessel V, shown in cross-section. While the disclosure herein is made with reference largely to the introducer sheath 100, and components thereof, the disclosure may also apply to a repositioning sheath, as will be discussed further herein. In some embodiments, the introducer sheath 100 may be used for facilitating the passage of various medical devices, such as a catheter or a blood pump as will be described further herein, through the introducer sheath 100 and into the blood vessel V. Hence, in some instances the introducer sheath 100 may be referred to as a large bore introducer sheath. The introducer sheath 100 includes a proximal end region 106 proximate a proximal end of the introducer sheath 100 and a distal end region 108 proximate a distal end of the introducer sheath 100 that is opposite the proximal end region 106. A body portion 110 of the introducer sheath 100 extends between the proximal end region 106 and the distal end region 108, and the body portion 110 defines a lumen 112 of the introducer sheath 100. The introducer sheath 100 includes a proximal opening (not shown) adjacent the proximal end region 106 and a distal opening 109 adjacent the distal end region 108, with the lumen 112 extending from the proximal opening to the distal opening 109. The introducer sheath 100, or components thereof, may be formed by various materials, such as polymeric and/or metallic materials. In some instances, the introducer sheath 100, such as the elongate shaft of the introducer sheath 100, may include an additional surface coating, such as but not limited to, silicone, PET, or other applicable polymer.

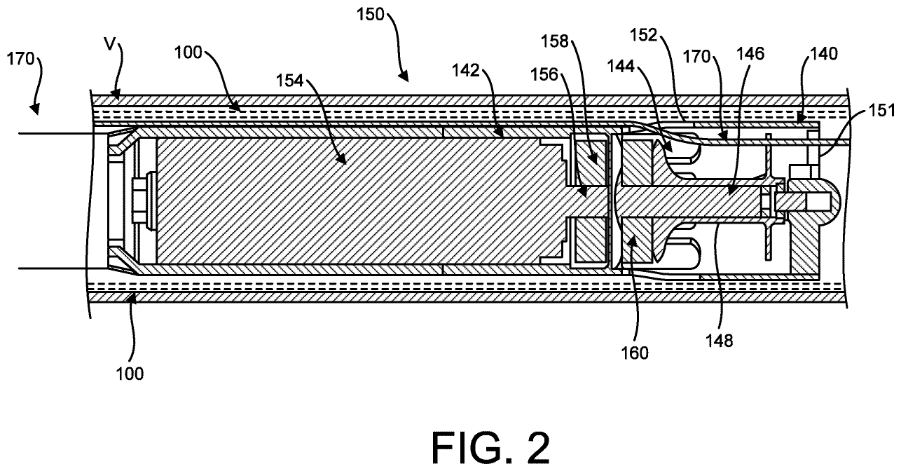

A hemostasis valve hub 120 (hereinafter “hub 120” for brevity) may be provided at the proximal end region 106 to provide access to the lumen 112 of the introducer sheath 100. The hub 120 may be configured for hemostasis by, for example, helping to prevent blood from leaking out of the introducer sheath 100 during use. For example, a medical device 170, such as a catheter or blood pump, may be inserted through the hub 120 and lumen 112 of the introducer sheath 100 and into the blood vessel V, and the hub 120 may maintain hemostasis between the medical device 170, the introducer sheath 100, and the external surroundings. In some embodiments, the medical device 170, may include and/or be coupled to a blood pump 150, shown in FIG. 2. After insertion of the medical device, fixation of the axial and radial position of the medical device 170 may be desired to ensure that the medical device 170 (and any device coupled thereto) is maintained in a proper position during use. It may also be desired for the medical personnel to reposition the medical device 170 after insertion. As such, the hub 120 may include a tightening port 130, provided with or attachable thereto, that provides for the fixation of the medical device 170 with respect to the hub 120 and blood vessel V, as will be described further herein. The hub 120 and tightening port 130 may allow for the repositioning of the medical device 170 with respect to the hub 120 and the blood vessel V.

FIG. 2 illustrates a cross-sectional view of the body portion 110 of the introducer sheath 100 of FIG. 1 upon insertion of a medical device, illustratively a blood pump 150, into the introducer sheath 100. As noted above, the medical device 170 of FIG. 1 may be coupled to or include the blood pump 150, with the medical device 170 extending outside the blood vessel V and the introducer sheath 100. The blood pump 150 may be advanced through the blood vessel V and positioned in a target location, such as a target cardiac location (e.g., the left ventricle), via the introducer sheath 100. The blood pump 150 may generally include an impeller assembly housing 140 and a motor housing 142. In some embodiments, the impeller assembly housing 140 and the motor housing 142 may be integrally or monolithically constructed. In other instances, the impeller assembly housing 140 and the motor housing 142 may be separate components. The impeller assembly housing 140 carries an impeller assembly 144 therein. The impeller assembly 144 may include an impeller shaft 146 and an impeller 148 that rotate relative to the impeller assembly housing 140 to drive blood through the blood pump 150. More specifically, the rotation of the impeller 148 causes blood to flow from a blood inlet 151 formed on the impeller assembly housing 140, through the impeller assembly housing 140, and out of a blood outlet 152 formed on the impeller assembly housing 140. In some embodiments, the impeller shaft 146 and the impeller 148 may be integrally formed, whereas, in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may be coupled to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly 144. More specifically, in embodiments incorporating the impeller shaft 146, the impeller shaft 146 and the impeller 148 are configured to rotate with the driven magnet 160. In other embodiments, the motor 154 may be coupled to the impeller assembly 144 via other components. While the introducer sheath 100 is illustrated above with the use of the blood pump 150, various other medical devices may be used in conjunction with the introducer sheath 100 and the hemostasis valve hub 120.

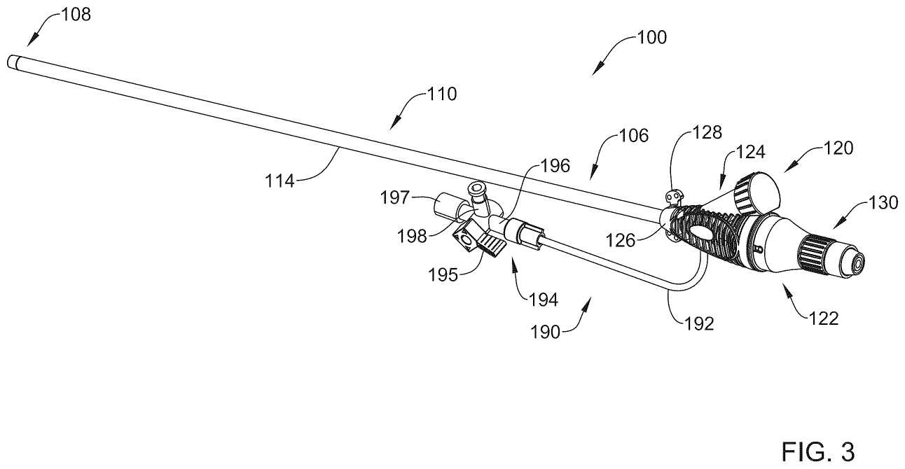

FIG. 3 is a perspective view of the introducer sheath 100 of FIG. 1 in which further details of the introducer sheath 100 are illustrated. For example, the introducer sheath 100 includes the hub 120, an elongate shaft 114 extending distally from the hub and defining the body portion 110 of the sheath 100. The sheath 100 may also include a flush line 190 extending from the hub 120. The flush line 190 may include a tubular member 192 extending from the hub 120 and in fluid communication with a lumen of the hub 120. The tubular member 192 may extend to a stopcock 194, such as a three-way stopcock. The stopcock 194 may include a first leg 196 coupled to the tubular member 192, a second leg 197, a third leg 198, and a lever 195 that is rotatable to selectively open/close fluid access between the first, second and/or third legs 196, 197, 198. Each of the legs 196, 197, 198 may include a connector, such as a luer connector, as desired.

The hub 120 may also include a strain relief 126 configured to provide a transition flexibility along the proximal end region 106. The strain relief 126 may include a body attached to a main body of the hub 120, as will be described further herein. The strain relief 126 may include one or more suture pads 128 extending outward therefrom. For example, the strain relief 126 may include first and second suture pads 128 extending from opposite sides of the strain relief 126. The suture pads 128 may facilitate securing the hub 120 against the patient once the introducer sheath 100 has been positioned in the blood vessel of the patient. For example, each suture pad 128 may include at least one opening extending therethrough for receiving a suture used to suture the hub 120 to the skin of the patient.

The suture pads 128 may be angled at an acute angle relative to the longitudinal axis of the sheath 100. For instance, the suture pads 128 may be angled toward the distal end region 108 of the sheath 100 such that the free ends of the suture pads 128 are positioned distal of the base ends of the suture pads 128. In some instances, the angle between the suture pads 128 and the longitudinal axis of the sheath 100 may be about 30 degrees to about 90 degrees, about 35 degrees to about 85 degrees, about 40 degrees to about 80 degrees, about 45 degrees to about 75 degrees, about 50 degree to about 70 degrees, or about 55 degrees to about 65 degrees. In some instances, the angle between the suture pads 128 and the longitudinal axis of the sheath 100 may be about 35 degrees, 40 degrees, 45 degrees, 50 degrees, 55 degrees, or 60 degrees.

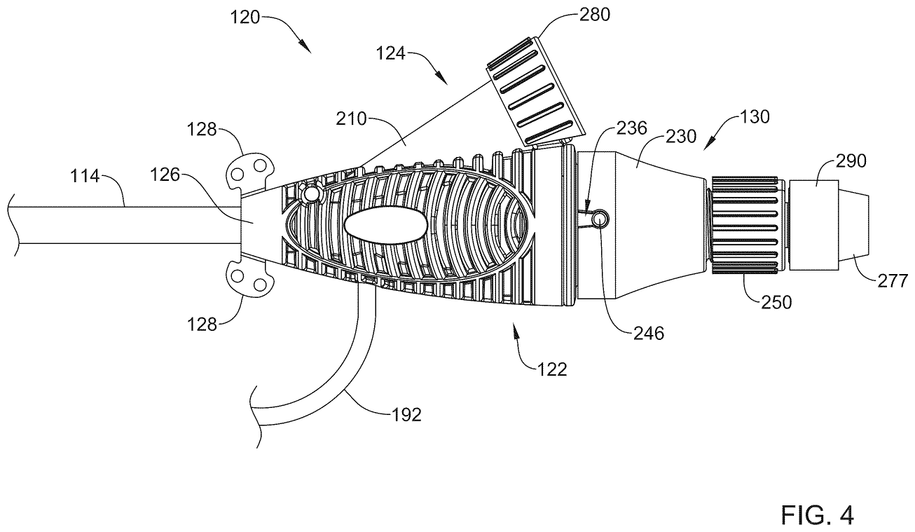

In some instances, as shown in FIG. 4, the strain relief 126 may be formed as a monolithic portion of the hub body 210. However, in other instances, the strain relief 126 may be formed as a separate component attached to a distal end portion of the hub body 210. For example, a distal end region of the hub body 210 may extend into the strain relief 126 and form a snap fit or interference fit therewith. For instance, the distal end region of the hub body 210 may include an annular rim configured to engage (e.g., form a snap fit) with a mating recess formed in the interior of the strain relief 126.

The hub 120 may include a main port 122 and a side port 124 extending from the main port 122. In some instances, the side port 124 may extend at an acute angle or a perpendicular angle from the main port 122. The main port 122 and/or the side port 124 may provide access to one or more lumens extending through the body portion 110 of the sheath 100. In some instances, the main port 122 may be the tightening port 130, as discussed above.

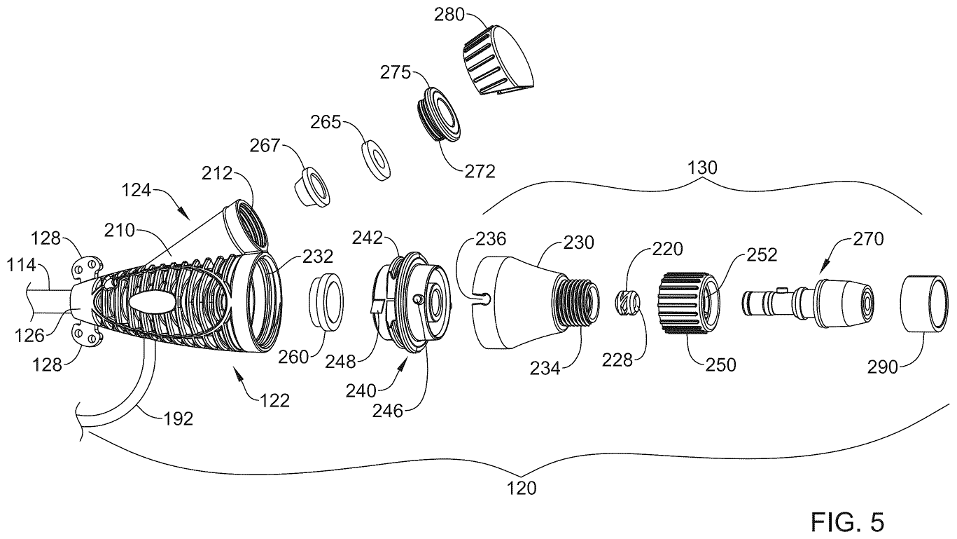

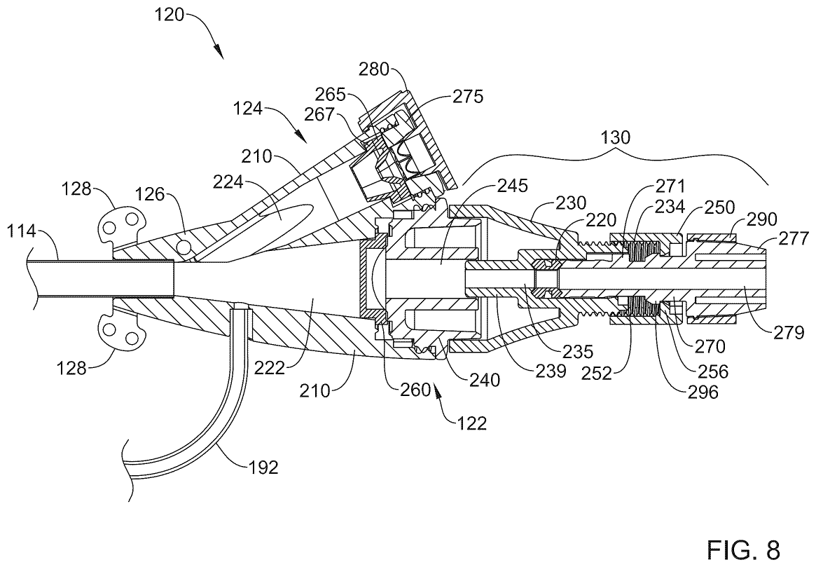

Further details of the components of the hub 120 are shown in the side view of FIG. 4, the exploded side view of FIG. 5, and the cross-sectional view of FIG. 8. The main port 122 of hub 120 may include a hub body 210. In some instances, the hub body 210 may be a molded, one-piece structure. For example, the hub body 210 may be molded from a polymeric material. In other instances, the hub body 210 may be formed of two or more components attached together. In some instances, the hub body 210 may also include or be connected to the hub body of the side port 124.

The side port 124 may include a passage 224 extending therethrough. For example, hub body 210 may define the passage 224. The side port 124 may also include one or more seals positioned along the passage 224 of the side port 124. For instance, the side port 124 may include a first elastomeric seal 265 positioned along the passage 224 of the side port 124 and a second elastomeric seal 267 positioned along the passage 224 of the side port 124. In some instances, the first elastomeric seal 265 may be juxtaposed with the second elastomeric seal 267. However, in other instances, the first elastomeric seal 265 may be spaced apart from the second elastomeric seal 267, if desired. Each of the elastomeric seals 265, 267 may be a slit valve (e.g., a cross-slit valve), a dome valve, a duckbill valve, or any other desired valve configured to seal around an elongate shaft of a medical device when passed therethrough. In some instances, the elastomeric seal 265 and/or the elastomeric seal 267 may include one or more slits (e.g., crossing slits) extending entirely through the seal wall and/or one or more slits (e.g., crossing slits) extending only partially through the seal wall. For instance, the elastomeric seal 265, 267 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seals 265, 267 may be formed of any desired flexible material, such as silicone, polyurethane, etc.

As shown in FIG. 8, the first elastomeric seal 265 may be positioned proximal of the second elastomeric seal 267 with a rim of the first elastomeric seal 265 juxtaposed with the rim of the second elastomeric seal 267 with a central portion of the first elastomeric seal 265 extending distally into the interior of the second elastomeric seal 267. Other arrangements of the first and second elastomeric seals 265, 267 are also contemplated.

The side port 124 may also include a side port lid 275 connectable to the hub body 210. For example, the side port lid 275 may include threading 272 (e.g., male threading), shown in FIG. 5, configured to engage (e.g., threadingly engage) with mating threading 212 (e.g., female threading) of the hub body 210. In other instances, the side port lid 275 may be connected to the hub body 210 in another fashion, if desired. The elastomeric seal 265 and/or the elastomeric seal 267 may be housed within the hub body 210 and/or in the side port lid 275 such that the elastomeric seals 265, 267 are captured between a surface of the hub body 210 and a surface of the side port lid 275. In some instances, the elastomeric seal 265 may be compressed against the elastomeric seal 267 when the side port lid 275 is coupled to the hub body 210.

The side port 124 may also include a side port cap 280 configured to threadably engage the side port lid 275. For example, the side port cap 280 may include internal threads configured to threadably engage external threads in the side port lid 275. Removal of the side port cap 280 may allow access to the elastomeric seal 265 and/or elastomeric seal 267 such that a medical device may be passed through the elastomeric scal 265 and/or elastomeric seal 267 into the side port passageway 224.

The main port 122, may include a passage 222 extending therethrough. For example, the hub body 210 may define the passage 222. The passage 224 of the side port 124 may converge with the passage 222 of the main port 122 within the hub body 210. The passage 222 and/or the passage 224 may be in fluid communication with the lumen 112 of the sheath 100 extending to the distal opening 109 (See FIG. 1).

The main port 122 may include a first seal and a second seal spaced apart from the first seal along a length of the main port 122. For example, the main port 122 may include a compressible seal (e.g., Tuohy seal) 220 and an elastomeric seal 260 spaced apart from the compressible seal 220. The elastomeric seal 260 may be a slit valve (e.g., a cross-slit valve), a dome valve, a duckbill valve, or any other desired valve configured to seal around an elongate shaft of a medical device when passed therethrough. In some instances, the elastomeric seal 260 may include one or more slits (e.g., crossing slits) extending entirely through the seal wall and/or one or more slits (e.g., crossing slits) extending only partially through the seal wall. For instance, the elastomeric seal 260 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seal 260 may be formed of any desired flexible material, such as silicone, polyurethane, etc.

The elastomeric seal 260 may be coupled to the hub body 210 via the main port lid 240. For example, the elastomeric seal 260 may be positioned within the interior of the hub body 210 and the main port lid 240 may then be coupled to the hub body 210, capturing the elastomeric seal 260 between a surface of the hub body 210 and a surface of the main port lid 240. In some instances, the elastomeric seal 260 may be compressed between a surface of the hub body 210 and a surface of the main port lid 240 when the main port lid 240 is coupled to the hub body 210. The main port lid 240 may be coupled to the hub body 210 in any desired fashion. For example, the main port lid 240 may include threading 242 (e.g., male threading), shown in FIG. 5, configured to engage (e.g., threadingly engage) with mating threading 232 (e.g., female threading) of the hub body 210. In other instances, the main port lid 240 may be connected to the hub body 210 in another fashion, if desired. For example, the main port lid 240 may include an engagement feature (e.g., a lip, rim, recess, notch, etc.) configured to engage (e.g., form a snap fit) with a mating engagement feature (e.g., lip, rim, recess, notch, etc.) of the hub body 210. In addition or alternative to forming a threaded connection or snap fit, the main port lid 240 may be adhesively bonded to the hub body 210, or otherwise secured to the hub body 210.

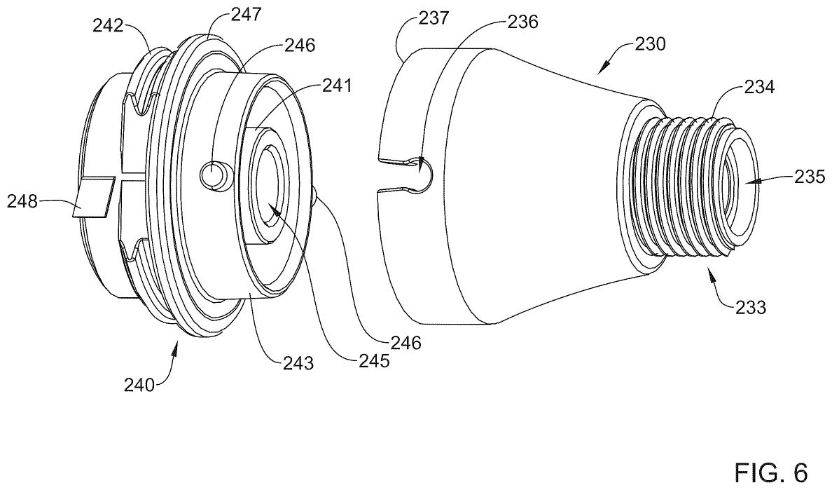

In some instances, the main port lid 240 may include an anti-rotation feature configured to prevent unscrewing of the main port lid 240 from the hub body 210 once connected thereto. For example, the main port lid 240 may include a tab 248 (or a plurality of tabs) configured to engage a portion of the hub body 210. The tab 248 may be configured to permit rotation of the main port lid 240 in a first direction (e.g., clockwise) to threadably connect and tighten the main port lid 240 onto the hub body 210, but prevent rotation of the main port lid 240 in a second direction (e.g., counter-clockwise) to prevent unthreading or loosening the main port lid 240 from the hub body 210. As shown in FIG. 6, in some instances the tab 248 may be located distal of the threading 242 of the main port lid 240. However, other arrangements of the tab 248, or other anti-rotation feature, are contemplated.

The main port 122 may include a tightening port 130, which may be a detachable assembly, configured to be attachable to and/or detachable from the hub body 210 of the hub 120. In some instances, the tightening port 130 may be attachable to and/or detachable from the hub body 210 of the hub 120 intraoperatively, as described later herein. The tightening port 130 may be constructed of several components, to provide a hemostasis valve assembly. For example, the tightening port 130 may include the compressible seal 220, a tightening port hub 230, a pusher 270, and a lock nut 250. Rotation of the lock nut 250 may actuate the pusher 270 toward/away from the compressible seal 220 to adjust the size of the opening 228 through the compressible seal 220. As described further herein, the compressible seal 220 may be movable between an open state, allowing a medical device to pass through the opening 228, and a closed state, sealing the compressible seal 220 around the medical device. The compressible seal 220 may be formed of any desired flexible material, such as silicone, polyurethane, etc.

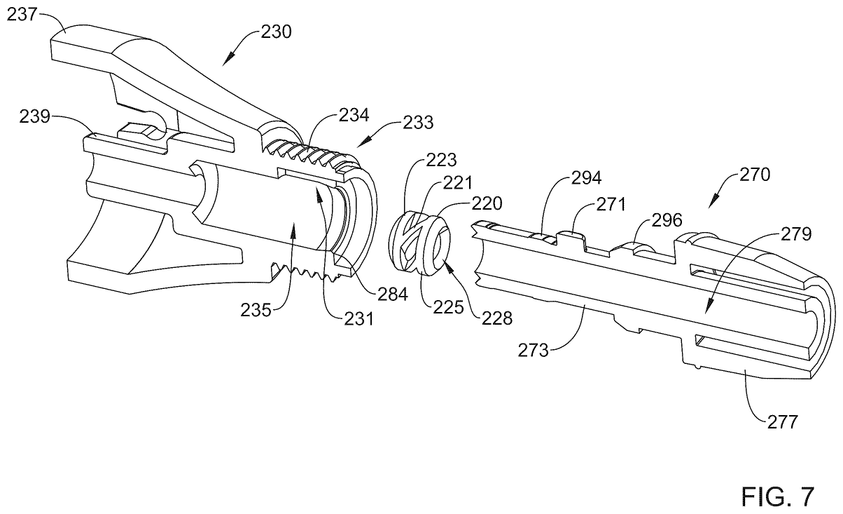

Referring to FIG. 7, the compressible seal 220 may include a distal annulus of material 223, a proximal annulus of material 225, and one or more, or a plurality of helical or angled ribs 221 extending between the distal annulus of material 223 and the proximal annulus of material 225. Such a construction may provide the compressible seal 220 with sufficient structural support, while permitting axial and radial compression of the compressible seal 220 when a compressive force is exerted thereon. Other constructions of the compressible seal 220 are also contemplated.

As shown in FIG. 8, the compressible seal 220 may be positioned in the interior of the tightening port hub 230 with the opening 228 of the compressible seal 220 axially aligned with the passageway 222 of the main port 122. A distal end region of the pusher 270 may be positioned in the interior of the tightening port hub 230 with a distal end surface of the pusher 270 juxtaposed with a proximal end surface of the compressible seal 220. For instance, the compressible seal 220 and the distal end region of the pusher 270 may be positioned within a lumen 235 of the tightening port hub 230 with the compressible seal 220 positioned between a shoulder of the lumen 235 and the distal end surface of the pusher 270. As shown in FIG. 7, the pusher 270 may include an enlarged spindle 277 and a stem 273 extending distal from the enlarged spindle 277. A lumen 279 may extend through an entire length of the pusher 270, including through the stem 273 and the spindle 277. The distal end region of the stem 273 may be inserted into the lumen 235 of the tightening port hub 230 while the spindle 277 may have a larger diameter than the lumen 235, preventing insertion of the spindle 277 in the lumen 235. The lumen 279 extending through the pusher 270 may be axially aligned with the lumen 228 of the seal 220 and the lumen 235 of the tightening port hub 230.

Referring additionally to FIG. 7, the pusher 270 may include one or more tabs 271 extending outward therefrom configured to be positioned in one or more channels 231 formed in the tightening port hub 230. For example, the pusher 270 may include a tab 271 aligned with and axially moveable along the channel 231 of the tightening port hub 230. Positioning the tab 271 in the channel 231 may prevent rotational movement of the pusher 270 relative to the tightening port hub 230 and/or the compressible seal 220 while permitting axial movement of the pusher 270 relative to the tightening port hub 230 to move the pusher 270 toward and/or away from the compressible seal 220. The channel 231 may allow the tab 271 of the pusher 270 a range of axial travel between a distalmost position proximate the distal edge of the channel 231 and a proximalmost position. The tab 271 may be located on an intermediate region of the pusher 270 such that a distal end region of the pusher 270 may extend distal of the tab 271 and a proximal end region of the pusher 270 may extend proximal of the tab 271.

Furthermore, the tightening port hub 230 may include an interference fit member, such as an inward extending rim 284 extending around a circumference of the lumen 235 of the tightening port hub 230, configured to form an interference fit with an interference fit member, such as an outward extending rim 294 extending around a circumference of the pusher 270. In some instances, the outward extending rim 294 may be positioned distal of the tab 271, in the distal end region of the pusher 270. When the pusher 270 is assembled with the tightening port hub 230, the rim 294 (or other interference fit member) of the pusher 270 may be located distal of the rim 284 (or other interference fit member) of the tightening port hub 230 to inhibit decoupling the pusher 270 from the tightening port hub 230. For instance, the placement of the rim 294 distal of the rim 284, with the distal end region of the pusher 270 inserted into the lumen 235 of the tightening port hub 230, may allow for a range of axial movement between the pusher 270 and the tightening port hub 230, but prevent the distal end region of the pusher 270 from being removed from the lumen 235 of the tightening port hub 230. For example, the rim 294 of the pusher 270 may engage the rim 284 at the proximalmost position of the tab 271 in the channel 231.

The tightening port hub 230 may be configured to be detachably coupled to the main port lid 240 to permit the tightening port 130 to be selectively coupled to and decoupled from the main port 122. Referring additionally to FIG. 6, the main port lid 240 may include an engagement structure configured to engage with and/or interlock with the tightening port hub 230. For example, the main port lid 240 may include first and second tabs 246 extending outward therefrom configured to be positioned in first and second slots 236, respectively, formed in the tightening port hub 230 while a distal rim 237 of the tightening port hub 230 is juxtaposed with an annular rim 247 of the main port lid 240. Furthermore, a stem 239 (shown in FIG. 7), through which the lumen 235 of the tightening port hub 230 extends through, may be inserted into the lumen 245 of the main port lid 240 when the tightening port hub 230 is coupled to the main port lid 240, shown in FIG. 8. Accordingly, the lumen 235 of the tightening port hub 230 may be axially aligned with and in communication with the lumen 245 extending through the main port lid 240.

In some embodiments, the slots 236 may be generally key-hole shaped, having an enlarged hole for receiving the tabs 246 therein and an open channel extending from the enlarged hole to the distal rim 237 such that the tabs 246 can be inserted into the enlarged hole of the slots 236 via the open channel. The open channel may have a width less than a diameter of the enlarged hole and the tabs 246 such that the tabs 246 snap-fit into the enlarged holes in the slots 236. Other locking arrangements are contemplated to selectively couple the tightening port hub 230 to the main port lid 240. For example, a bayonet-style locking mechanism may be used. In one example, the tabs 246 may be inserted into L-shaped slots forming in the tightening port hub 230 and then the tightening port hub 230 may be rotated relative to the main port lid 240 to lock the tabs 246 in the slots.

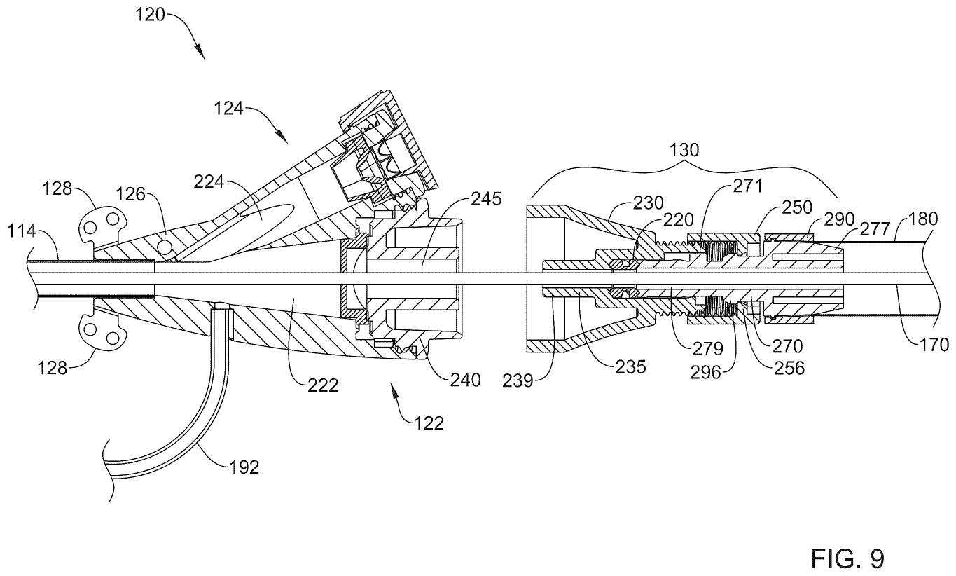

The tightening port 130 may include a coupling interface configured to couple a sterile sleeve 180 (shown in FIG. 9) to the tightening port 130. For example, a collar 290, also called a sleeve gripper, may interact with an enlarged spindle 277 of the pusher 270 to provide a coupling interface configured to grip the sterile sleeve 180 therebetween. For example, an end portion of a sterile sleeve 180 (shown in FIG. 9), may be slid over or placed around (e.g., surround) the enlarged spindle 277 of the pusher 270. The spindle 277 may include a tapered or chamfered surface to facilitate sliding the enlarged spindle 277 of the pusher 270 into the lumen of the sterile sleeve 180. The collar 290 may be placed around the portion of the sterile sleeve 180 surrounding the spindle 277 to thereby compress the sterile sleeve 180 between the collar 290 and the spindle 277 of the pusher 270.

The lock nut 250 may be assembled such that the lock nut 250 surrounds the stem 273 of the pusher 270. The lock nut 250 may include internal threading 252 threadably engaged with external threading 234 provided on an exterior of a proximal portion of the tightening port hub 230. For instance, the tightening port hub 230 may include a threaded stem 233 having the external threading 234 formed thereon. The threaded stem 233 may be a generally cylindrical portion of the tightening port hub 230 extending proximal of a frustoconical body port of the tightening port hub 230.

As shown in the cross-sectional view of FIG. 8, the components of the tightening port 130 may form a hemostasis valve. Accordingly, rotating the lock nut 250 in a first rotational direction causes the lock nut 250 to travel distally relative to the tightening port hub 230 (and thus the hub body 210), and rotating the lock nut 250 in a second, opposite rotational direction causes the lock nut 250 to travel proximally relative to the tightening port hub 230 (and thus the hub body 210). As shown in FIG. 8, the lock nut 250 may include an internal rim 256 configured to engage the rim 296 of the pusher 270. In some instances, the rim 296 may be located on the stem 273 of the pusher 270 such that the internal rim 256 is located axially between the rim 296 and the enlarged spindle 277 of the pusher 270. For example, a distal facing surface of the internal rim 256 may engage a proximally facing surface of the rim 296 of the pusher 270. The internal rim 256 may extend continuously around the lock nut 250 and/or the rim 296 may extend continuously around the pusher 270. Thus, the distally facing surface of the internal rim 256 may maintain engagement with the rim 296 of the pusher 270 as the lock nut 250 is rotated through one or more full revolutions. As the lock nut 250 is rotated, and thus travels axially relative to the tightening port hub 230 and the hub body 210, the lock nut drives the pusher 270 in an axial direction. For example, as the lock nut 250 is rotated in a first rotational direction relative to the tightening port hub 230 and the hub body 210, the lock nut 250 travels distally, urging the pusher 270 to travel distally relative to the tightening port hub 230 and the hub body 210 to exert a compressive force on the compressible scal 220 to thereby reduce the diameter of the opening 228 through the compressible seal 220. As the lock nut 250 is rotated in a second rotational direction relative to the tightening port hub 230 and the hub body 210, the lock nut 250 travels proximally, permitting the pusher 270 to travel proximally relative to the tightening port hub 230 and the hub body 210 to reduce and/or remove the compressive force on the compressible seal 220 to thereby increase the diameter of the opening 228 through the compressible seal 220.

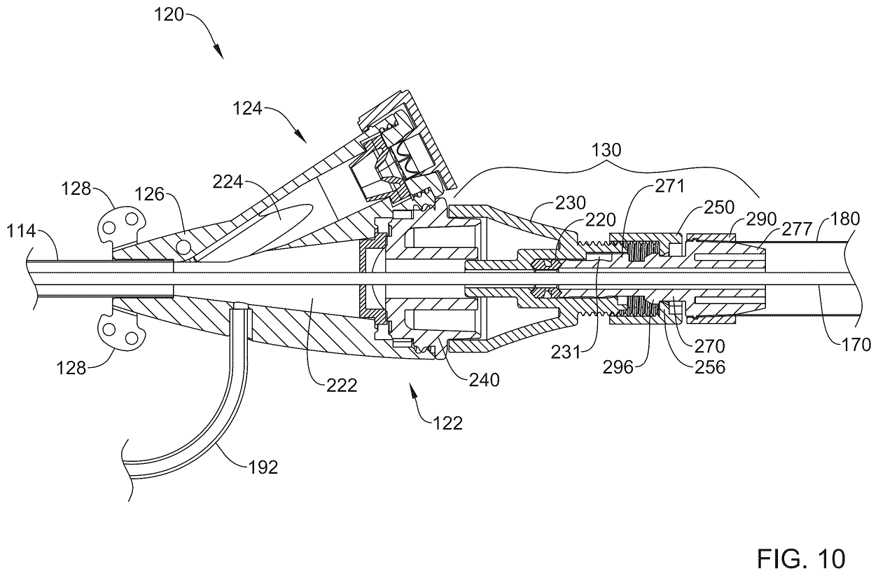

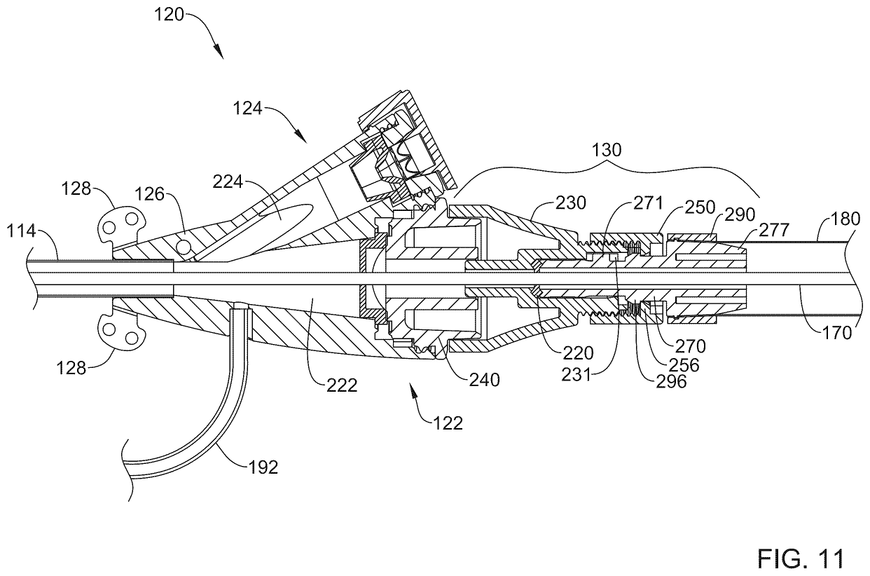

FIG. 9 is a cross-sectional view of the introducer sheath of FIG. 3, with the tightening port 130 provided with a medical device and detached from the hub body 210 of the main port 122 of the hub 120 of the introducer sheath 100. FIG. 10 is a cross-sectional view of the introducer sheath 100 of FIG. 3 with the tightening port 130 attached to the hub body 210, via connection to the main port lid 240, and the medical device 170 passing through the hemostasis valve provided by the tightening port 130 in an open position. FIG. 11 is a cross-sectional view of the introducer sheath of FIG. 3 with the tightening port 130 attached to the hub body 210, via connection to the main port lid 240, and with the medical device 170 passing through the hemostasis valve provided by the tightening port 130 in a closed position. In some instances, the tightening port 130 (e.g., hemostasis port) may be configured to accommodate a medical device having a diameter of 12 F or more, 14 F or more, 15 For more, 16 F or more, or 17 F or more. In other words, the compressible seal 220 and the elastomeric seal 260 may be configured to permit a medical device having a diameter of 12 F or more, 14 F or more, 15 F or more, 16 F or more, or 17 F or more, to pass therethrough while maintaining hemostasis.

In use, the tightening port 130 may be provided preassembled with the medical device with the sterile sleeve 180 coupled to the tightening port 130 (e.g., with the sterile sleeve 180 secured to the pusher 270 via the collar 290) and the elongate shaft of the medical device 170 extending through the lumen 279 of the pusher 270, through the lumen 228 of the compressible seal 220, and through the lumen 235 of the tightening port hub 230. With the tightening port 130 detached from the hub body 210, the elongate shaft of the medical device 170 may be intraoperatively inserted into the lumen 245 of the main port lid 240 and through the elastomeric seal 260 into the passageway 222 of the main port 122. For example, the elongate shaft of the medical device 170 may be passed through an opening of the elastomeric seal 260 and cause the elastomeric seal 260 to flex and/or deform to permit the elongate shaft of the medical device 170 to be advanced therethrough. The elastomeric seal 260 may seal around an outer perimeter of the elongate shaft of the medical device 170 and substantially prevent blood from escaping from the main port 122. The elongate shaft of the medical device 170 may be further advanced through the elongate shaft 114 of the sheath 100 into the body of the patient for a diagnostic and/or therapeutic procedure.

As shown in FIG. 11, once the elongate shaft of the medical device 170 is positioned at a desired location within the vasculature of the patient, the compressible seal 220 may be compressed or tightened around the perimeter of the elongate shaft of the medical device 170 to prevent blood from leaking past the medical device 170 and out of the main port 122 and/or lock the elongate shaft of the medical device 170 from axial movement relative to the hub 120. For instance, as discussed above, the lock nut 250 may be rotated to distally advance the pusher 270, which in turn exerts a compressive force against the compressible seal 220, reducing the diameter of the opening 228 through the compressible seal 220, to seal the compressible seal 220 around the elongate shaft of the medical device 170.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An introducer sheath, comprising:
a valve hub defining a main port and a side port; and
an elongate shaft extending from the valve hub;
wherein the main port includes:
a hub body;
a seal disposed within the hub body,
a main port lid attached to the hub body;
a tightening port hub attachable to the main port lid;
a compressible seal disposed within a lumen of the tightening port hub;
a pusher at least partially positioned within the lumen of the tightening port hub and slidably movable relative thereto; and
a lock nut surrounding the pusher, the lock nut threadably engaged with the tightening port hub such that rotation of the lock nut moves the pusher axially toward and/or away from the compressible seal.

2. The introducer sheath of claim 1, wherein the pusher includes a flange extending radially from a body of the pusher.

3. The introducer sheath of claim 2, wherein an internal rim of the lock nut engages the flange.

4. The introducer sheath of claim 1, wherein the main port lid is threadably engaged with the hub body of the main port.

5. The introducer sheath of claim 1, wherein the main port lid includes first and second tabs extending radially therefrom.

6. The introducer sheath of claim 5, wherein the tightening port hub includes first and second slots for receiving the first and second tabs, respectively.

7. The introducer sheath of claim 1, wherein the lock nut is threadably engaged with a threaded stem of the tightening port hub.

8. The introducer sheath of claim 1, wherein the pusher includes an anti-rotation tab slidably disposed in a channel of the tightening port hub.

9. The introducer sheath of claim 1, wherein a distal end region of the pusher that is positioned within the lumen of the tightening port hub includes an enlarged diameter rim, wherein the enlarged diameter rim has a larger diameter than a portion of the lumen located proximal thereof.

10. The introducer sheath of claim 1, further comprising a sleeve gripper surrounding a proximal end region of the pusher.

11. A medical device assembly, comprising:
a medical device having an elongate shaft;
a tightening port attachable to a hub of an introducer sheath, the tightening port including:
a tightening port hub;
a compressible seal disposed within a lumen of the tightening port hub;
a pusher at least partially positioned within the lumen of the tightening port hub and slidably movable relative thereto; and
a lock nut surrounding the pusher, the lock nut threadably engaged with the tightening port hub such that rotation of the lock nut moves the pusher axially toward and/or away from the compressible seal; and
a sterile sleeve surrounding the elongate shaft such that the elongate shaft is moveable axially relative to the sterile sleeve through a lumen of the compressible seal, a distal end of the sterile sleeve coupled to the tightening port.

12. The medical device assembly of claim 11, wherein the distal end of the sterile sleeve is secured to the pusher.

13. The medical device assembly of claim 12, wherein a spindle of the pusher is inserted into a lumen of the sterile sleeve and a collar surrounds the distal end of the sterile with the distal end of the sterile sleeve clamped between the collar and the spindle.

14. The medical device assembly of claim 11, wherein a distal end region of the pusher that is positioned within the lumen of the tightening port hub includes an enlarged diameter rim, wherein the enlarged diameter rim has a larger diameter than a portion of the lumen located proximal thereof.

15. The medical device assembly of claim 11, wherein the pusher includes a flange extending radially from a body of the pusher and an internal rim of the lock nut engages the flange.

16. A method of inserting a medical device into a body of a patient, comprising:
intraoperatively inserting an elongate shaft of the medical device through an elastomeric seal in a hub of an introducer sheath and into a vasculature of the patient;
after inserting the elongate shaft through the elastomeric seal, coupling a tightening port to the hub of the introducer sheath.

17. The method of claim 16, wherein the elongate shaft extends through a compressible seal of the tightening port as the elongate shaft of the medical device is inserted through the elastomeric seal in the hub of the introducer sheath and into the vasculature of the patient.

18. The method of claim 17, wherein the tightening port includes:
a tightening port hub;
a compressible seal disposed within a lumen of the tightening port hub;
a pusher at least partially positioned within the lumen of the tightening port hub and slidably movable relative thereto; and
a lock nut surrounding the pusher, the lock nut threadably engaged with the tightening port hub such that rotation of the lock nut moves the pusher axially toward and/or away from the compressible seal.

19. The method of claim 18, further comprising:
tightening the compressible seal around the elongate shaft of the medical device with the lock nut after coupling the tightening port to the hub of the introducer sheath.

20. The method of claim 17, further comprising a sterile sleeve having a distal end coupled to the tightening port, wherein the sterile sleeve extends proximally from the tightening port and surrounds the elongate shaft such that the elongate shaft is moveable axially relative to the sterile sleeve.
